(19) United States
(12) Patent Application Publication
(10) Pub. No.: US 2012/0093909 A1
(43) Pub. Date: Apr. 19, 2012

(54) Ceramic Coatings and Applications Thereof

(76) Inventor: Ahmed El-Ghannam, Charlotte,, NC (US)

(21) Appl. No.: 13/378,829

(22) PCT Filed: Jun. 17, 2010
(86) PCT No.: PCT/US10/38959

§ 371 Date: Dec. 16, 2011

Related U.S. Application Data
(60) Provisional application No. 61/187,779, filed on Jun. 17, 2009.

(51) Int. Cl. A61K 9/00; C25D 15/00; A61K 33/42; B82Y 5/00

(52) U.S. Cl. 424/423; 424/602; 204/471; 204/509; 977/931

(57) ABSTRACT

In one aspect, the present invention provides coated metal substrates which, in some embodiments, demonstrate one or more advantageous chemical and/or mechanical properties.

### RELATED APPLICATION DATA

The present application claims priority pursuant to 35 U.S.C. §119(e) to U.S. Provisional Patent Application Ser. No. 61/187,779, filed Jun. 17, 2009 which is hereby incorporated by reference in its entirety.

### FIELD OF THE INVENTION

The present invention relates to ceramic coatings and, in particular, to biocompatible ceramic coatings.

### BACKGROUND OF THE INVENTION

An estimated 11 million people in the United States have at least one medial device implant. Generally, two types of implants, fixation devices and artificial joints, are used in orthopedic treatments and oral-maxillofacial procedures.

Tissue integration between bone and an orthopedic implant is essential for sufficient fixation and longevity of the implant. As a result, about 80% of fracture fixation devices require adjuvant grafting. Currently, autograft material is preferentially used. Autograft material, however, presents certain difficulties including donor site morbidity, limited donor site bone supply, anatomical and structural problems as well as elevated levels of resorption during healing.

In view of these difficulties, various materials, such as hydroxyapatite [HA, Ca₁₀(PO₄)₆(OH)₂], have been applied to implant surfaces for improving in vivo response and performance of the implant. Nevertheless, limitations in the use of HA as a metal implant coating arise due to instability and failure at the HA/metal interface and/or reduced bioactivity resulting from high processing temperatures employed during plasma spraying of HA.

### SUMMARY

In one aspect, the present invention provides coated metal substrates which, in some embodiments, demonstrate one or more advantageous chemical and/or mechanical properties. In some embodiments, coated metal substrates described herein are suitable for use as implants in one or more orthopedic and/or dental applications.

In some embodiments, the present invention provides a composition comprising a metal substrate and a coating adhered to a surface of the metal substrate, the coating comprising electrophoretically deposited and sintered composite particles, the composite particles comprising a silica component and a calcium phosphate component, wherein the coating has an adhesion strength of at least about 30 MPa. In some embodiments, the coating has an adhesion strength of at least about 45 MPa. Moreover, in some embodiments, the coating has a substantially uniform thickness.

In another aspect, the present invention provides dispersions. In some embodiments, a dispersion comprises a continuous phase and a dispersed phase, the dispersed phase comprising composite particles, the composite particles comprising a silica component and calcium phosphate component, wherein the particles have a zeta potential of at least about −30 mV. In some embodiments, the composite particles have a zeta potential of at least about −40 mV.

In some embodiments, the continuous phase of a dispersion described herein comprises water. In some embodiments, the continuous phase of a dispersion comprises one or more alcohols. Additionally, in some embodiments, the continuous phase of a dispersion comprises a mixture of water and one or more alcohols.

In another aspect, the present invention provides methods of producing a coated metal substrate. In some embodiments, a method of producing a coated metal substrate comprises providing the metal substrate, providing a dispersion comprising a continuous phase and a dispersed phase comprising composite particles, the composite particles comprising a silica component and a calcium phosphate component, wherein the particles have a zeta potential of at least about −30 mV. The metal substrate is immersed in the dispersion and a charge is induced on a surface of the metal substrate. The composite particles are deposited on the surface of the metal substrate to provide the coating. In some embodiments, the metal substrate is provided as an electrode. In one embodiment, for example, the metal substrate is provided as an anode.

In a further aspect, the present invention provides methods of treating a patient. In some embodiments, a method of treating a patient comprises providing an implant and positioning the implant at an implant site of the patient, the implant comprising a metal substrate and a coating adhered to a surface of the metal substrate, the coating comprising electrophoretically deposited and sintered composite particles, the composite particles comprising a silica component and a calcium phosphate component, wherein the coating has an adhesion strength of at least about 30 MPa.

These and other embodiments are described in greater detail in the detailed description which follows.

### BRIEF DESCRIPTION OF THE DRAWINGS

FIG. 1 illustrates variation of the zeta potential of dispersed composite particles with pH according to one embodiment of the present invention.

FIG. 2 illustrates variation of dispersion conductivity with pH according to one embodiment of the present invention.

FIG. 3 illustrates variation of the zeta potential of dispersed composite particles with the chemical identity of the continuous phase according to one embodiment of the present invention.

FIG. 4 illustrates variation in dispersion conductivity with chemical identity of the continuous phase according to one embodiment of the present invention.

FIG. 5 is an scanning electron micrograph (SEM) of a coated metal substrate according to one embodiment of the present invention.

FIG. 6 is an x-ray diffraction (XRD) analysis of a coating according to one embodiment of the present invention.

FIGS. 7(a)-(c) are SEM images of a coating according to one embodiment of the present invention after immersion in PBS solution.

FIG. 8 illustrates the weight change of a metal coated substrate according to one embodiment of the present invention after immersion in PBS solution.

### DETAILED DESCRIPTION

The present invention can be understood more readily by reference to the following detailed description, examples and drawings and their previous and following descriptions. Elements, apparatus and methods of the present invention, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

### I. Coated Metal Substrates

In one aspect, the present invention provides coated metal substrates which, in some embodiments, demonstrate one or more advantageous chemical and/or mechanical properties. In some embodiments, coated metal substrates described herein are suitable for use as implants in one or more orthopedic and/or dental applications.

In some embodiments, the present invention provides a composition comprising a metal substrate and a coating adhered to the metal substrate, the coating comprising electrophoretically deposited and sintered composite particles, the composite particles comprising a silica component and a calcium phosphate component, wherein the coating has an adhesion strength of at least about 30 MPa. In some embodiments, the coating has an adhesion strength of at least about 35 MPa. The coating, in some embodiments, has an adhesion strength of at least about 40 MPa or at least about 45 MPa. In some embodiments, the coating has an adhesion strength ranging from about 30 MPa to about 50 MPa. As described further herein, the adhesion strength of a coating of the present invention is measured according to ASTM F1147-05, Standard Test Method for Tension Testing of Calcium Phosphate and Metallic Coatings.

A coating comprising electrophoretically deposited and sintered composite particles, in some embodiments, has a uniform or substantially uniform thickness. Moreover, a coating described herein, in some embodiments, has a uniform or substantially uniform thickness of at least about 1 μm. In some embodiments, a coating described herein has uniform or substantially uniform thickness up to about 50 μm. In some embodiments, a coating described herein has a uniform or substantially uniform thickness within any of the ranges set forth in Table I.

```
TABLE I
Coating Thickness
  1 μm-50 μm
  2 μm-20 μm
  5 μm-15 μm
  5 μm-10 μm
1 μm-7 μm
2 μm-6 μm
3 μm-5 μm
10 μm-15 μm
15 μm-50 μm
15 μm-50 μm
>50 μm
```

In some embodiments, a coating described herein comprising electrophoretically deposited and sintered composite particles is continuous or substantially continuous over the surface of the metal substrate. In being continuous or substantially continuous over the surface of the metal substrate, the coating does not display breaks or discontinuities revealing patches of uncoated metal substrate. In some embodiments, a break or discontinuity for determining the continuous nature of a coating described herein is at least 10 μm in size. In some embodiments, a break or discontinuity is at least 20 μm in size. Moreover, in some embodiments, a break or discontinuity is at least one order of magnitude larger than an average particle size of the coating. Additionally, in some embodiments, a coating described herein is dense and free or substantially free of porosity.

Turning now to specific components, a coating described herein comprises electrophoretically deposited and sintered composite particles, the composite particles comprising a silica component and a calcium phosphate component. In some embodiments, a composite particle comprises silica in an amount ranging from about 20 weight percent to about 80 weight percent. A composite particle, in some embodiments, comprises silica in an amount ranging from about 40 weight percent to about 60 weight percent. In some embodiments, a composite particle comprises silica in an amount of at least about 50 weight percent.

In some embodiments, a silica component of a composite particle described herein comprises one or more silica polymorphs. In some embodiments, for example, a silica component comprises α-quartz, β-quartz or mixtures thereof. In some embodiments, a silica component comprises α-tridymite, β-tridymite or mixtures thereof. Additionally, in some embodiments, a silica component comprises α-cristobalite, β-cristobalite or mixtures thereof.

In some embodiments, a composite particle comprises a calcium phosphate in an amount ranging from about 20 weight percent to about 80 weight percent. A composite particle, in some embodiments, comprises a calcium phosphate in an amount ranging from about 40 weight percent to about 60 weight percent. In some embodiments, a composite particle comprises a calcium phosphate in an amount of at least about 50 weight percent.

In some embodiments, a calcium phosphate component of a composite particle described herein comprises one or more types of calcium phosphate. In some embodiments, for example, a calcium phosphate component comprises α-tricalcium phosphate, β-tricalcium phosphate, tetracalcium phosphate, hydroxyapatite, bicalcium phosphate, calcium pyrophosphate (β-Ca₂P₂O₇), dibasic calcium phosphate or rhenanite (β-NaCaPO₄) or mixtures thereof.

In some embodiments, the composite particles of a coating described herein have an average size ranging from about 5 nm to about 15 μm. In some embodiments, composite particles of a coating described herein have an average size ranging from about 10 nm to about 5 μm or from about 50 nm to about 1 μm.

Composite particles of a coating described herein, in some embodiments, have a bimodal average particle size distribution. In some embodiments, for example, a portion of the composite particles of the coating have a first average particle and a portion of the composite particles have a second average particle size, the second average particle size different from the first average particle size. In some embodiments, the second average particle size is at least an order of magnitude larger than the first average particle size.

In some embodiments, the first average particle size ranges from about 5 nm to about 1 μm. In some embodiments, the first average particle size ranges from about 20 nm to about 800 nm or from about 50 nm to about 500 nm. In some embodiments, the second average particle size ranges from about 2 μm to about 20 μm. In some embodiments, the second average particle size ranges from about 3 μm to about 15 μm or from about 5 μm to about 10 μm.

In some embodiments wherein a coating described herein comprises a bimodal composite particle size distribution, composite particles of the first average particle size are at least partially disposed in spaces or voids existing between composite particles of the second average particle size.

In addition to a coating comprising electrophoretically deposited and sintered composite particles comprising a silica component and a calcium phosphate component, a composition described herein comprises a metal substrate to which the coating is adhered. In some embodiments, the metal substrate comprises a metal or metal alloy. In some embodiments, a metal comprises a transition metal. In some embodiments, a metal alloy comprises a transition metal alloy.

A metal substrate, in some embodiments, comprises titanium or a titanium metal alloy. In some embodiments, a titanium alloy comprises aluminum, vanadium or nickel or mixtures thereof. In one embodiment, for example, a metal substrate comprises Ti-6Al-4V. In some embodiments, a titanium alloy comprises nickel. In one embodiment, for example, a titanium alloy comprises a nickel-titanium alloy (e.g., nitinol). In some embodiments, a transition metal alloy comprises cobalt or chromium or combinations thereof. In some embodiments, a metal substrate comprises a metal oxide layer disposed between the coating and the metal or metal alloy of the substrate.

A metal substrate, in some embodiments, is porous. A metal substrate, in some embodiments, for example, displays bulk porosity throughout the substrate. In some embodiments, a substrate displays surface porosity with no or substantially no bulk porosity. In some embodiments, a porous metal substrate has a pore structure operable to provide framework or scaffold for new tissue and/or bone growth to occur. In some embodiments, pores of a porous metal substrate have diameters ranging from about 100 μm to about 1 mm. In some embodiments wherein the metal substrate is porous, a coating described herein comprising composite particles adheres to walls of the pores and does not occlude or substantially occlude the pores of the metal substrate. In some embodiments, a coating described herein comprising composite particles has a uniform or substantially uniform thickness over complex surfaces such as pore walls, cusps or other geometrical/topographical features of the metal substrate surface.

Moreover, a substrate can have any desired thickness not inconsistent with the objectives of the present invention. In some embodiments, the thickness of a metal substrate is determined according the application in which the coated metal substrate is to be used. In some embodiments, a metal substrate has thickness suitable for one or more orthopedic applications. A metal substrate, in some embodiments, has thickness suitable for one or more dental applications.

A metal substrate can have any desired shape. In some embodiments, the shape of a metal substrate is determined according to the application in which the coated metal substrate is to be used. In some embodiments, a metal substrate has plate shape, curved shape, spherical shape, elliptical shape, disc shape or a cylinder/rod shape. In some embodiments, a metal substrate has the shape of an anchoring device, such as a screw or a nail. In some embodiments, a metal substrate has the shape of an artificial joint or part thereof. An artificial joint, in some embodiments, comprises a hip or knee.

In some embodiments, a composition comprising a metal substrate and a coating adhered to the metal substrate, the coating comprising electrophoretically deposited and sintered composite particles, the composite particles comprising a silica component and a calcium phosphate component is operable to support deposition of hydroxyapatite on the coating when immersed in a physiologic solution such as phosphate buffered saline (PBS).

In some embodiments, a coating of a metal substrate described herein further comprises one or more antibiotic, antimicrobial or antiviral agents. Any desired antibiotic, antimicrobial or antiviral agent not inconsistent with the objectives of the present invention may be used for incorporation into or onto surfaces of a coating described herein. In some embodiments, an antibiotic, antimicrobial or antiviral agent is applied to a coating described herein in solution form. In some embodiments, an antibiotic, antimicrobial or antiviral agent is applied to a coating described herein in a gel form or a foam form. In some embodiments, an antibiotic, antimicrobial or antiviral agent is applied to a coating described herein in a polymeric carrier such as in a polymeric coating. Additionally, in some embodiments, an antibiotic, antimicrobial or antiviral agent may be grafted onto a surface of a coating described herein by one or more grafting techniques including radical polymerization or condensation reactions.

In some embodiments, an antibiotic comprises vancomycin. In some embodiments, for example, vancomycin can be applied to coatings of metal substrates described herein as a salt solution of vancomycin hydrochloride.

### II. Dispersions of Composite Particles

In another aspect, the present invention provides dispersions. In some embodiments, a dispersion comprises a continuous phase and a dispersed phase comprising composite particles, the composite particles comprising a silica component and a calcium phosphate component, wherein the composite particles have a zeta potential of at least about −30 mV. In some embodiments, the zeta potential of the composite particles is at least about −35 mV. In some embodiments, the zeta potential of the composite particles is at least about −40 mV. Additionally, in some embodiments, the zeta potential of the composite particles ranges from about −30 mV to about −50 mV.

Moreover, in some embodiments, a dispersion described herein has a conductivity of less than about 30 μS/cm. In some embodiments, a dispersion described herein has a conductivity of less than about 25 μS/cm or less than about 20 μS/cm. A dispersion described herein, in some embodiments, has a conductivity of less than about 15 μS/cm or less than about 10 μS/cm. In some embodiments, a dispersion described herein has a conductivity of less than about 5 μS/cm. In some embodiments, a dispersion described herein has a conductivity of less than about 3 μS/cm.

Turning now to specific components, a dispersion described herein comprises a continuous phase. In some embodiments, a continuous phase comprises water. Water, in some embodiments, comprises deionized water. In some embodiments, a continuous phase of a dispersion comprises one or more alcohols. Any alcohol not inconsistent with the objectives of the present invention can be used. In some embodiments, an alcohol comprises a monohydric alcohol, a polyhydric alcohol or a alicyclic alcohol or mixtures thereof. In some embodiments, a monohydric alcohol comprises methanol, ethanol, propanol, isopropanol, butanol, or pentanol or mixtures thereof.

Additionally, in some embodiments, a continuous phase comprises a mixture of water and one or more alcohols. A continuous phase comprising a mixture of water and one or more alcohols can have any desired weight percent of alcohol not inconsistent with the objectives of the present invention. In some embodiments, for example, a continuous phase comprises 50 wt % alcohol and 50 wt % water.

As described herein, the dispersed phase comprises composite particles, the composite particles comprising a silica component and a calcium phosphate component. In some embodiments, the composite particles of the dispersed phase can have any of the properties recited for the same in Section I hereinabove. In some embodiments, for example, the composite particles of the dispersed phase have composition and average particle sizes, including bimodal average particle sizes, as described in Section I hereinabove.

A dispersion described herein, in some embodiments, comprises composite particles in an amount of at least about 1% (w/v). In some embodiments, an aqueous dispersion comprises composite particles in an amount of at least about 2% (w/v). In some embodiments, an aqueous dispersion comprises composite particles in an amount of at least about 5% (w/v) or at least about 10% (w/v). Additionally, in some embodiments, an aqueous dispersion comprises composite particles in an amount ranging from about 0.5% (w/v) to about 10% (w/v). In some embodiments, an aqueous dispersion comprises composite particles in an amount ranging from about 2% (w/v) to about 5% (w/v).

Furthermore, in some embodiments, a dispersion described herein has a pH ranging from about 3 to about 9. In some embodiments, a dispersion described herein has a pH ranging from about 6 to about 8.

### III. Methods of Producing a Coated Metal Substrate

In another aspect, the present invention provides methods of producing a coated metal substrate. In some embodiments, a method of producing a coated metal substrate comprises providing the metal substrate, providing a dispersion comprising a continuous phase and a dispersed phase comprising composite particles, the composite particles comprises a silica component and a calcium phosphate component, wherein the particles have a zeta potential of at least about −30 mV. The metal substrate is immersed in the dispersion and a charge is induced on a surface of the metal substrate. The composite particles are deposited on the surface of the metal substrate to provide the coating.

In some embodiments of a method of producing a coated metal substrate, the dispersion comprising a continuous phase and a dispersed phase comprising composite particles can have any of the properties recited in Section II hereinabove. Moreover, in some embodiments, composite particles of the dispersion comprising a silica component and calcium phosphate component and/or the metal substrate can have any of the properties recited for the same in Section I hereinabove.

Surfaces of a metal substrate, in some embodiments, are passivated prior to deposition of a coating described herein comprising composite particles. In some embodiments, passivation of a metal substrate surface provides a metal oxide layer on which a coating described herein is deposited.

In some embodiments, for example, a titanium substrate or titanium alloy substrate is immersed in nitric acid (HNO₃) or NaOH for a sufficient amount of time prior to immersion in a dispersion for deposition of a coating comprising composite particles, the composite particles comprising a silica component and a calcium phosphate component. Passivation of the titanium substrate or titanium alloy substrate with HNO₃, in some embodiments, provides a titanium oxide layer on which the coating is deposited. In some embodiments, surfaces of a metal substrate are roughened or abraded prior to deposition of a coating described herein. In some embodiments, surfaces of a metal substrate are roughened by sanding or other mechanical abrading. In some embodiments, surfaces of a metal substrate are roughened by chemical etching, plasma etching, ion etching or electromagnetic etching.

In some embodiments, the metal substrate is provided as an electrode. In one embodiment, for example, the metal substrate is provided as an anode onto which positive charge is induced for the deposition of composite particles described herein. In some embodiments, deposition of a coating described herein is conducted at a voltage ranging from about 20V to 130V. In some embodiments, deposition of a coating described herein is conducted at a voltage ranging from about 30V to about 120V.

Deposition of composite particles on a surface of a charged metal substrate can be administered for any time period not inconsistent with the objectives of the present invention. In some embodiments, deposition of composite particles on a surface of a charged metal substrate is administered for a time period ranging from about 30 seconds to about 600 seconds.

In some embodiments, a method of producing a coated metal substrate further comprises sonicating the dispersion prior to deposition of composite particles on a surface of a charged metal substrate. In some embodiments, sonication of a dispersion comprising composite particles can be administered for any amount of time not inconsistent with the objectives of the present invention. In some embodiments, a dispersion is sonicated for a time period ranging from about 30 seconds to about 60 minutes. In some embodiments, sonicating a dispersion reduces particle sizes and/or reduces aggregation of the dispersed composite particles.

A method of producing a coated metal substrate, in some embodiments, further comprises subjecting the coating to a heat treatment. In some embodiments, a heat treatment comprises heating the deposited coating at a temperature ranging from about 300° C. to about 900° C. In some embodiments, a heat treatment comprises heating the deposited coating at a temperature of at least about 600° C. In some embodiments, a heat treatment comprises heating the deposited coating at a temperature of up to about 900° C. In some embodiments, a heat treatment of the coating is conducted at a temperature sufficiently low so as to not alter or substantially alter the crystalline structure of the metal substrate. By avoiding alteration of the crystalline structure of the metal substrate, a heat treatment, in some embodiments, does not change or compromise the mechanical and/or chemical properties of the substrate.

In some embodiments, the deposited coating is heated in an inert atmosphere such as under argon or nitrogen. Subjecting a coating to heat treatment, in some embodiments, sinters the composite particles of the coating. In some embodiments, sintering the composite particles of the coating results in a dense coating with no or substantially no porosity.

### IV. Methods of Treating a Patient

In some embodiments, a method of treating a patient comprises providing an implant and positioning the implant at an implant site of the patient, the implant comprising a metal substrate and a coating adhered to a surface of the metal substrate, the coating comprising electrophoretically deposited and sintered composite particles, the composite particles comprising a silica component and a calcium phosphate component, wherein the coating has an adhesion strength of at least about 30 MPa.

In some embodiments of a method of treating a patient, providing an implant comprises any of methods described in Section III hereinabove. Moreover, a coated metal substrate of an implant can have any of the properties recited in Section I hereinabove. Additionally, in some embodiments, an implant site of a patient comprises an area of fractured, diseased or otherwise damage bone tissue.

Some embodiments of the present invention are further illustrated by the following non-limiting examples.

### Example I

### Preparation of Composite Particles Comprising a Silica Component and Calcium Phosphate Component

Composite particles comprising various weight percents of silica and calcium phosphate as set forth in Table II were prepared according to the following procedure. Appropriate ratios of dicalcium phosphate CaHPO₄.2H₂O and silica were placed in a polyethylene bottle and mixed on a roller mixer for 24 h. The resulting mixture was moistened with 0.1 M NaOH and placed in a teflon mold of 10 mm diameter×10 mm height. The mixture was dried at room temperature and subsequently sintered in air at 850° C. for 2 hours. The sintered silica-calcium phosphate mixture was ground in a roller jar mill and separated mechanically on stainless steel sieves to provide composite particles having a silica component and a calcium phosphate component. Composite particles less than 600 μm were further ground in a PM 100 planetary ball mill from Retsch Technology of Newtown, Pa. for a period of 24-34 hours to produce nanosized composite particles.

TABLE II
Composite Particle Compositions

| Composite Particle Sample | Composite Particle Composition |
|---|---|
| SCPC25 | Calcium Phosphate—75 wt % |
| | Silica—25 wt % |
| SCPC50 | Calcium Phosphate—50 wt % |
| | Silica—50 wt % |
| SCPC75 | Calcium Phosphate—25 wt % |
| | Silica—75 wt % |

### Example II

### Dispersion pH, Zeta Potential and Conductivity

The affect of pH on the zeta potential of composite particles described herein was determined by providing dispersions of composite particles SCPC25, SCPC50 and SCPC75 in a 50% ethanol/water continuous phase of varying pH according to Table III. The pH of the dispersions was varied using NH₄OH or HNO₃. A sample of each dispersion [3.0 ml of 0.1% (w/v) SCPC dispersion] was analyzed for zeta potential and conductivity using a solvent resistant electrode connected to a ZetaPALS of Brookhaven Instruments Corp. of Holtsville, N.Y. The zeta potential was determined by measuring the electrophoretic mobility (μ) of the SCPC particles. The Smoluchowski equation was used to calculate SCPC's zeta potential (ζ).

μ=εζ/η

where: μ is the electrophoretic mobility, ε is the electric permittivity of the medium and η is the viscosity. To calculate conductivity from conductance values, the conductance of standard 1 mM KCl of known conductivity (137 μS/cm at 24° C.) was measured as 377 μS. Cell constant was then calculated based on the following relationship:

Conductivity=Cell constant*Conductance

The cell constant was found to be 0.36 cm⁻¹. The conductance values obtained were multiplied by the cell constant to obtain the conductivity values.

TABLE III
Dispersion Composition and pH

| Dispersion Sample | |
|---|---|
| (50% Ethanol | |
| Continuous Phase) | pH |
| SCPC25 | 2 |
| SCPC25 | 3 |
| SCPC25 | 4 |
| SCPC25 | 5 |
| SCPC25 | 6 |
| SCPC25 | 7 |
| SCPC25 | 8 |
| SCPC25 | 9 |
| SCPC50 | 2 |
| SCPC50 | 3 |
| SCPC50 | 4 |
| SCPC50 | 5 |
| SCPC50 | 6 |
| SCPC50 | 7 |
| SCPC50 | 8 |
| SCPC50 | 9 |
| SCPC75 | 2 |
| SCPC75 | 3 |
| SCPC75 | 4 |
| SCPC75 | 5 |
| SCPC75 | 6 |
| SCPC75 | 7 |
| SCPC75 | 8 |
| SCPC75 | 9 |

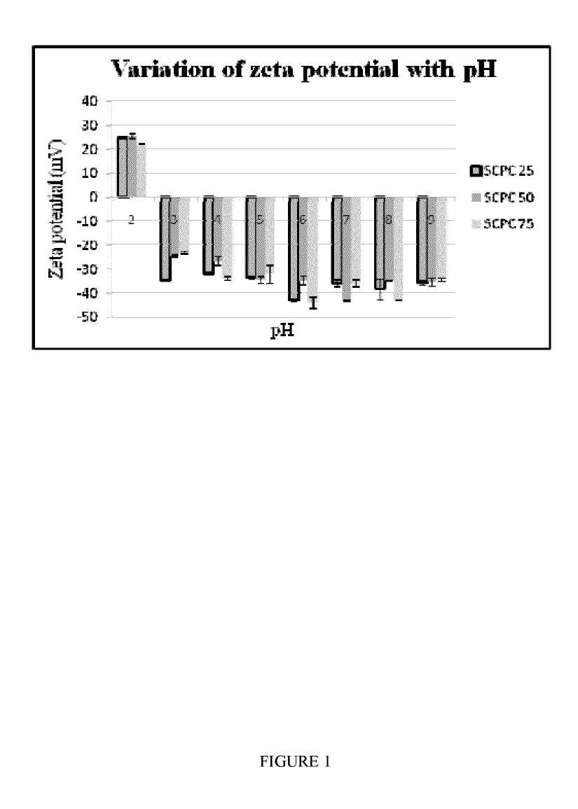

FIG. 1 illustrates the variation of the zeta potential of SCPC particles of different chemical compositions as a function of the pH of the continuous phase or suspending medium (50% ethanol). At pH 2, all SCPC samples acquired comparable positive zeta potential values in the range of 22-25 mV. However, at pH 3, all SCPC samples reversed the surface charge to be negative. The switch in the surface charge from positive to negative values indicated that the iso-electric point of SCPC in 50% ethanol occurred in the pH range 2-3, wherein the net charge carried by the SCPC particles was zero. SCPC25 had a significantly higher negative zeta potential (−35 mV) than SCPC50 and SCPC75 at pH 3 (p<0.05). SCPC50 and SCPC75 had comparable zeta potential values at the same pH. As the pH increased, the zeta potential of SCPC50 and SCPC75 increased in the pH range 3-5. However, minimal changes in the zeta potential of SCPC25 were observed in the same range of pH 3-5. All the three compositions acquired a maximum zeta potential value of (−43 mV) in the pH range of 6-8. While SCPC25 acquired the maximum zeta potential at pH 6, SCPC50 acquired its maximum potential at pH 7. SCPC75 acquired maximum zeta potential at pH 6 and continued to have similar potential at pH 8. Beyond pH 8, the zeta potential of all the three SCPC's decreased.

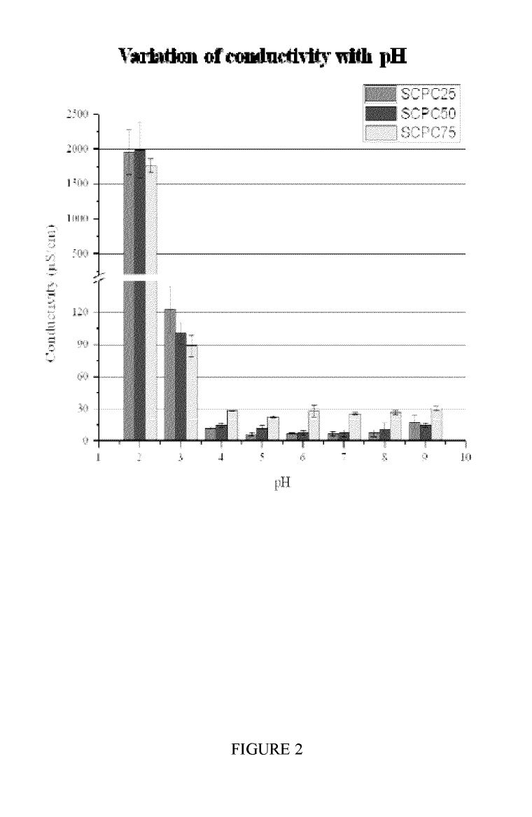

The conductivity of SCPC's of all compositions decreased sharply from (1768-1961 μS/cm) at pH 2 to (89-123 μS/cm) at pH 3 as illustrated in FIG. 2. Moreover, while comparable values of conductivity of all the three compositions were measured at pH 2, the conductivity of SCPC25 was higher than that of SCPC50; the latter was higher than that of SCPC75 at pH 3. On the other hand at pH 4, the conductivity of all SCPC samples further decreased, however, the conductivity of SCPC75 was significantly higher (p<0.02) than that of SCPC50 or SCPC25. Minimal changes in conductivity of all SCPC samples were observed in the pH range 4-9.

### Example III

### Dispersion Continuous Phase, Zeta Potential and Conductivity

The affect of the chemical identity of the continuous phase on the zeta potential of composite particles described herein was determined by providing dispersions of composite particles SCPC25, SCPC50 and SCPC75 in various continuous phases according to Table IV.

TABLE IV
Dispersion Composition

| Dispersed | |
|---|---|
| Phase | Continuous Phase |
| SCPC25 | 100% Ethanol |
| SCPC25 | 50% Ethanol/Water (pH 7) |
| SCPC25 | 100% DI Water |
| SCPC50 | 100% Ethanol |
| SCPC50 | 50% Ethanol/Water (pH 7) |
| SCPC50 | 100% DI Water |
| SCPC75 | 100% Ethanol |
| SCPC75 | 50% Ethanol/Water (pH 7) |
| SCPC75 | 100% DI Water |

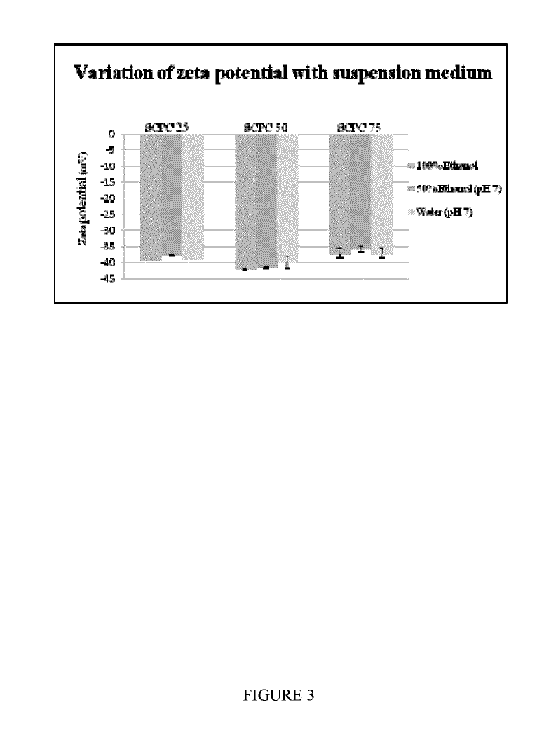

The zeta potential of the composite particles of each dispersion of Table IV was measured in accordance with the procedure set forth in Example II above. FIG. 3 illustrates the variation in zeta potential of SCPC25, SCPC50 and SCPC75 with variation in the chemical identity of the continuous phase. Each of the composite particle compositions acquired a higher zeta potential in pure ethanol than in continuous phases comprising deionized water. Moreover, SCPC50 acquired a significantly higher zeta potential than SCPC25 and SCPC75 (p<0.03).

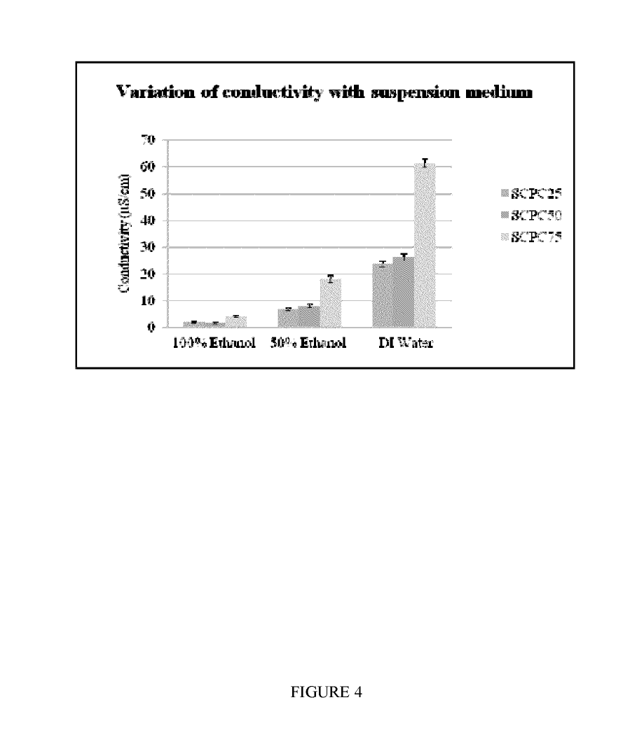

The conductivity of each dispersion of Table IV was determined in accordance with the procedure set forth in Example II above. FIG. 4 illustrates the conductivity of the SCPC compositions measured in 100% ethanol, 50% ethanol and DI water at pH 7. The conductivity of SCPC reached its maximum value in water and minimum value in 100% ethanol. In all suspension media, the conductivity of SCPC75 was higher than SCPC50 or SCPC25. Although, the conductivity of SCPC50 was higher than that of SCPC25, the difference was not statistically significant (p>0.9).

### Example IV

### Metal Coated Substrate

Metal coated substrates according to some embodiments of the present invention were prepared according to the following procedure. A metal substrate of a disc (1.3 cm diameter×0.5 cm thick) of medical grade Ti-6Al-4V (DePuy Inc. Warsaw, Ind.) was ground on a 400 grit silicon carbide abrasive pad (Leco Corp., St. Joseph, Mich.) and washed and cleaned according to ASTM standard protocols in DI water, phosphate-free detergent solution and acetone. The Ti alloy metal substrate was subjected to surface passivation before application of a coating comprising composite particles described herein. Passivation was conducted in 34% HNO₃ at 65° C. for 45 minutes followed by gentle washing in DI water. The passivation created a thin TiO₂ layer on the surface of the Ti alloy disc.

A dispersion comprising a continuous phase of ethanol and a dispersed phase SCPC50 particles was provided. The dispersion contained 5% (w/v) SCPC particles. The SCPC-ethanol dispersion was stirred for 15 minutes on a magnetic stirrer and subjected to ultrasonic agitation for 45 minutes with intermediate stirring to facilitate particle disaggregation.

The passivated Ti alloy disc was connected to a E3612A DC power supply and immersed in the SCPC-ethanol dispersion. Serving as the anode, the passivated Ti alloy disc was electrophoretically coated with SCPC50 composite particles at 50V for a time period of 180 seconds. During the electrophoretic deposition, the cathode was placed about 4.5 cm from the Ti alloy anode. The cathode was a Ti alloy disc having a 3.8 cm diameter and a 0.5 cm thickness. At the end of the coating process, the coated disc was removed and dried in a dessicator for 24 hours.

Subsequent to drying, the Ti alloy disc coated with SCPC50 composite particles was subjected to a thermal treatment. The coated Ti alloy disc was thermally treated in a Thermolyne muffle furnace (Themo Scientific, Dubuque, Iowa) at 800° C. for one hour. A controlled rate of heating and cooling (2-20° C./min) was used.

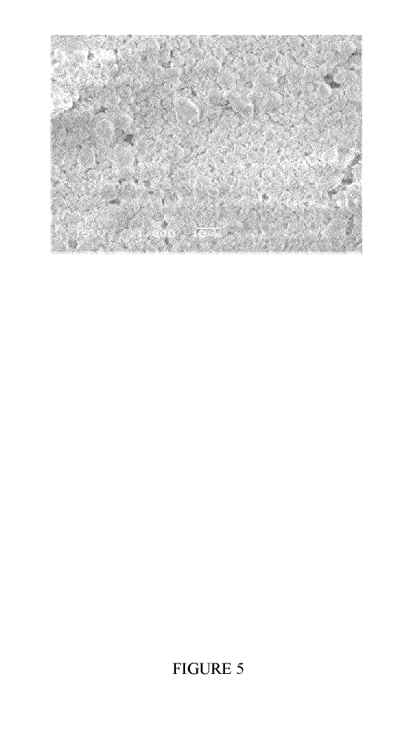

FIG. 5 is an SEM image of the coating of SCPC50 particles on the Ti alloy disc substrate after heat treatment. As illustrated in the SEM image, the coating comprising the SCPC50 particles is continuous over all or substantially all of the surface of the Ti alloy disc. Discontinuities or breaks, as defined herein, are not present in the SCPC50 coating. Moreover, the thickness of the SCPC coating was measured to be about 40 μm with minimal variation across the surface of the Ti alloy disc. Additionally, sintering between SCPC50 particles of the coating is illustrated in the SEM image of FIG. 5.

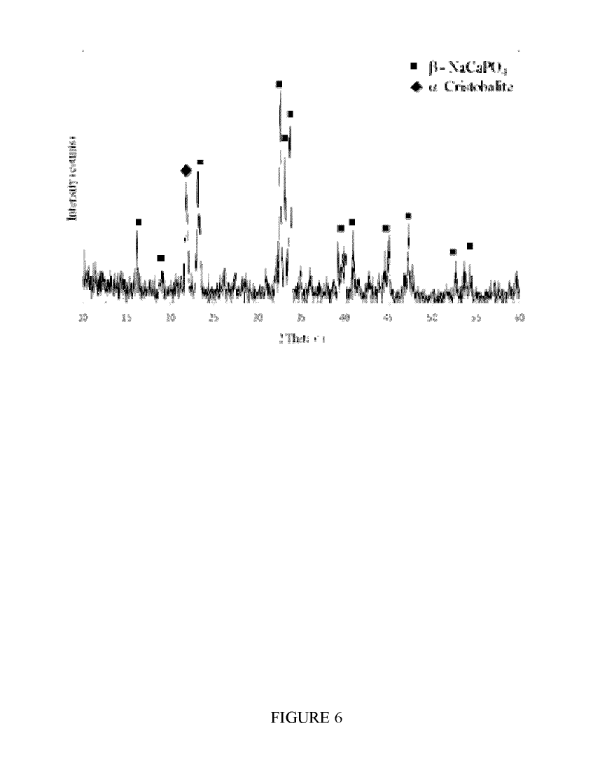

FIG. 6 is an x-ray diffraction (XRD) analysis of the SCPC50 coating. As illustrated in FIG. 6, the silica phase of the composite particles of the coating comprised α-cristobalite and the calcium phosphate phase of the composite particles of the coating comprised β-NaCaPO₄.

### Example V

### Coated Metal Substrate

A coated metal substrate was prepared in accordance with Example IV, the only difference being the electrophoretic deposition was administered for a time period of 120 seconds. The SCPC50 coating of the metal substrate was subjected to adhesion testing. For adhesion testing of the coating, the coated Ti alloy disc was glued to a Ti alloy cylinder of similar diameter with FM 1000 adhesive polymer (Cytec Industries, West Patterson, N.J.) and cured per ASTM F 1147-05 (Standard Test Method for Tension Testing of Calcium Phosphate and Metallic Coatings) for 1.5 hrs at 175 C under 25 psi pressure applied by means of calibrated temperature resistant spring. Adhesion strength of the coating was measured using an Instron testing machine at a crosshead rate of 2.54 mm/min until complete separation occurred, and the maximum load to fracture was calculated. The SCPC coating demonstrate a adhesion strength of 47±4 MPa.

### Example VI

### Immersion of Metal Coated Substrate in PBS

The SCPC50 coated Ti alloy disc of Example IV was immersed in 75 ml of PBS solution (Cellgro, Manassas, Va.) at 37° C. under orbital shaking 30 rpm. 5 ml of the supernatant was withdrawn every 24 hours and replaced with fresh PBS. At the conclusion of the 7 day period, the coated SCPC50 coated Ti alloy disc was recovered, dried at 37° C. in a hot air oven for 24 hours and analyzed by SEM. The weight of the SCPC50 coated Ti alloy disc was recorded before and after immersion in the PBS solution.

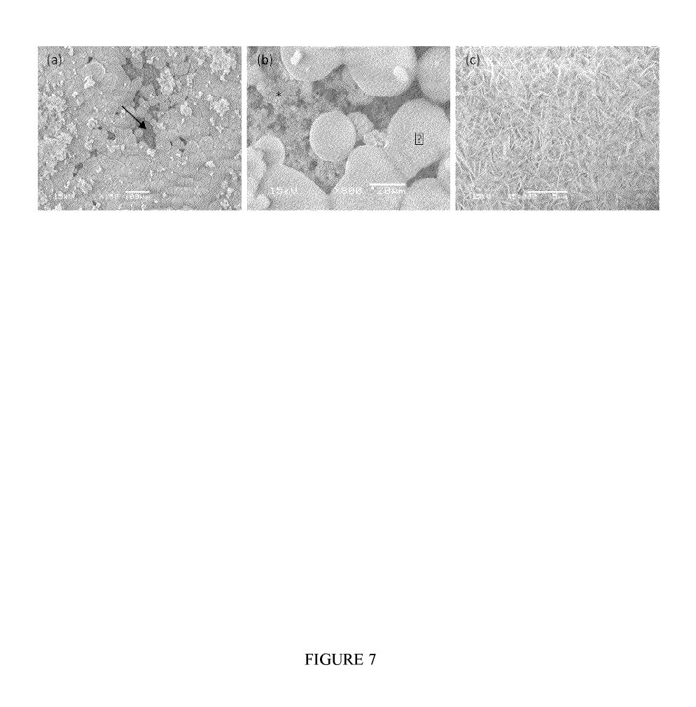

FIG. 7 provides SEM images of the SCPC coated Ti alloy disc after immersion in PBS at 37° C. for 7 days. An extensive hydroxyapatite layer could be seen uniformly spreading over the entire SCPC coated layer as illustrated in FIG. 7(a). FIG. 7(b) is a higher magnification SEM image displaying the intact SCPC layer (*) as well as the deposited hydroxyapatite layer (▪). Not only has the SCPC coated layer stimulated the formation of the apatite layer; it has also maintained its own integrity even after 7 days of immersion. FIG. 7(c) is a higher magnification SEM image of the apatite layer, demonstrating crystals of hydroxyapatite that are formed because of the back precipitation induced by the constituent ions of SCPC in PBS.

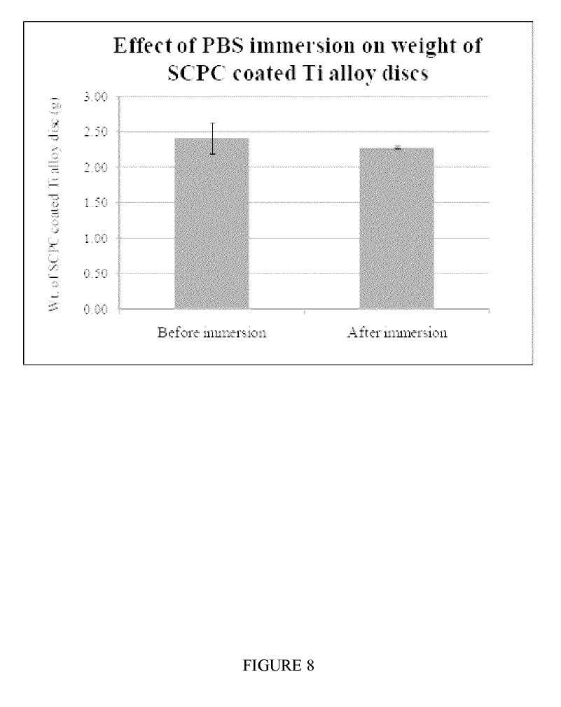

Weight analysis of the SCPC-coated Ti alloy disc before and after PBS immersion showed no significant weight loss at the end of the 7 day immersion period (FIG. 8). This indicates comparable rate of SCPC dissolution and back precipitation of the hydroxyapatite layer from the solution onto the material surface.

Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention.

That which is claimed is:

1. A composition comprising:
a metal substrate; and
a coating adhered to a surface of the metal substrate, the coating comprising electrophoretically deposited and sintered composite particles, the composite particles comprising a silica component and a calcium phosphate component, wherein the coating has an adhesion strength of at least about 30 MPa.

2. The composition of claim 1, wherein the composite particles have an average size ranging from about 20 nm to about 10 μm.

3. (canceled)

4. The composition of claim 1, wherein the composite particles have a bimodal average particle size distribution, the bimodal average particle size distribution having a first average particle size and a second average particle size different from the first average particle size.

5. The composition of claim 4, wherein the second average particle size is at least an order of magnitude larger than the first average particle size.

6. (canceled)

7. The composition of claim 1, wherein the silica component is present in the composite particle in an amount ranging from about 20 weight percent to about 80 weight percent.

8. (canceled)

9. (canceled)

10. The composition of claim 1, wherein the coating has a substantially uniform thickness.

11. (canceled)

12. The composition of claim 7, wherein the thickness of the coating is up to about 50 μm.

13. (canceled)

14. The composition of claim 1, wherein the coating is substantially continuous over the surface of the metal substrate.

15. The composition of claim 1, wherein the surface of the metal substrate comprises pores.

16. The composition of claim 15, wherein the coating does not substantially occlude the pores of the metal substrate.

17. The composition of claim 1, wherein the coating has an adhesion strength of at least about 35 MPa.

18. (canceled)

19. (canceled)

20. The composition of claim 1, wherein the metal substrate comprises a transition metal alloy.

21-24. (canceled)

25. The composition of claim 1, wherein the silica component comprises α-cristobalite.

26. The composition of claim 1, wherein the phosphate component comprises β-sodium-calcium phosphate.

27. A dispersion comprising:
a continuous phase; and a
dispersed phase comprising composite particles, the composite particles comprising a silica component and a calcium phosphate component, wherein the particles have a zeta potential of at least about −30 mV.

28. The dispersion of claim 27, wherein the composite particles have a zeta potential of at least about −35 mV.

29-31. (canceled)

32. The dispersion of claim 27, wherein the composite particles have a bimodal average size particle distribution, the bimodal average particle size distribution having a first average particle size and a second average particle size different from the first average particle size.

33-35. (canceled)

36. The dispersion of claim 27, wherein the continuous phase comprises water.

37. The dispersion of claim 27, wherein the continuous phase comprises a mixture of water and an alcohol.

38. (canceled)

39. The dispersion of claim 37, wherein the alcohol is present in an amount of up to about 50 weight percent.

40. (canceled)

41. The dispersion of claim 27, wherein the dispersion has a pH ranging from about 3 to 9.

42-44. (canceled)

45. The dispersion of claim 27, wherein the composite particles are present in an amount ranging from about 1% (w/v) to about 10% (w/v).

46. The dispersion of claim 27, wherein the dispersion has a conductivity less than about 30 μS/cm.

47. (canceled)

48. (canceled)

49. A method of producing a coated metal substrate comprising:
providing a metal substrate;
providing a dispersion comprising a continuous phase and a dispersed phase comprising composite particles, the composite particles comprising a silica component and a calcium phosphate component, wherein the particles have a zeta potential of at least about −30 mV;
immersing the metal substrate in the dispersion;
inducing a charge on a surface of the metal substrate; and
depositing the composite particles on the surface of the metal substrate to provide the coating.

50. The method of claim 49, wherein the metal substrate is provided as an electrode.

51. (canceled)

52. The method of claim 49, wherein the composite particles are deposited at a voltage ranging from about 30V to about 120V.

53. (canceled)

54. The method of claim 49 further comprising subjecting the coating to a heat treatment.

55. (canceled)

56. The method of claim 54, wherein the heat treatment comprises sintering the composite particles of the coating.

57. The method of claim 49 further comprising sonicating the aqueous dispersion prior to depositing the particles on the surface of the metal substrate.

58-60. (canceled)
